(19) United States
(12) Patent Application Publication
(10) Pub. No.: US 2009/0105788 A1
(43) Pub. Date: Apr. 23, 2009

(54) MINIMALLY INVASIVE NERVE MONITORING DEVICE AND METHOD

(75) Inventors: Stephen Bartol, Windsor (CA); Christopher Wybo, Royal Oak, MI (US)

Correspondence Address: RADER, FISHMAN & GRAUER PLLC, 39533 WOODWARD AVENUE, SUITE 140, BLOOMFIELD HILLS, MI, 48304-0610, US

(73) Assignee: Innovative Surgical Solutions, LLC

(21) Appl. No.: 12/040,515

(22) Filed: Feb. 29, 2008

Related U.S. Application Data
(60) Provisional application No. 60/980,996, filed on Oct. 18, 2007.

(51) Int. Cl. A61N 1/02

(52) U.S. Cl. 607/62

(57) ABSTRACT

A device includes a mechanical sensor configured to monitor at least one muscle for a response to a stimulus, and an indicator configured to provide feedback to a user based on at least a portion of an output of the mechanical sensor.

### CROSS REFERENCE TO RELATED APPLICATIONS

This application is a non-provisional of U.S. Provisional Application Ser. No. 60/980,996, entitled Minimally Invasive Nerve Monitoring Device and Method, filed Oct. 18, 2007 which is hereby incorporated herein by reference in its entirety.

### BACKGROUND INFORMATION

Nerve injury is a major risk during surgical procedures. Traditional surgical practices emphasize the importance of recognizing or verifying the location of nerves to avoid injuring them. Advances in surgical techniques include development of techniques including ever smaller exposures, such as minimally invasive surgical procedures, and the insertion of ever more complex medical devices. With these advances in surgical techniques, there is a corresponding need for improvements in methods of detecting and/or avoiding nerves.

Traditionally, the gold standard among nerve location has been direct visualization of a nerve. Direct visualization requires cutting through tissue surrounding the nerve to expose it, thereby allowing a surgeon to look at a nerve to ensure the nerve is not touched or damaged during a procedure.

Another conventional method used is nerve avoidance. By understanding human anatomy, and specifically where nerves should be within the body, a surgeon can work in the areas between the nerves, often referred to as “internervous planes of dissection,” thereby reducing the risk of damaging a nerve during a procedure.

While direct visualization and nerve avoidance can be effective procedures, they may be impractical for certain procedures. For instance, surgery generally involves a significant amount of blood and other fluids that may obscure a surgeon's view. It may be difficult to control fluid flowing in an area of interest, thereby making it difficult to see an exposed nerve, or to determine where adjacent nerves lie. Further, the physical limitations of human anatomy make these procedures impractical for many procedures. That is, the layout of the body is something of an inexact science, and often the location of nerves, much like muscle fibers and even entire organs, can vary between patients. In addition, each of these procedures may require additional operating time, and may necessitate cutting significant amounts of unaffected tissue, resulting in an increase in pain and scarring for a patient, as well as an increased healing time.

A more recent method of nerve monitoring involves electromyography (EMG). EMG is a technique used to measure electrical activity in a motor unit during static or dynamic activity, and to evaluate the health of nerves and corresponding muscles. A motor unit generally can be described as a motor neuron and the associated muscle fibers it innervates. EMG generally includes providing an electrical stimulus to a nerve, or to surrounding tissue, and analyzing an electrical response measured through metal electrodes. EMG requires that the metal electrodes maintain a consistent electrical connection with the innervated area in order to obtain a reading. In one common approach, the metal electrodes are needles which must be driven through the skin, directly into muscle tissue. In another approach, surface electrodes are used. Surface electrodes may require significant preparation of the skin, including first washing the skin, then cleaning the skin with alcohol, and debriding the skin with pumice stone or sand paper. Once the skin has been properly prepared, EMG surface electrodes must be covered with a conductive gel to improve the electrical connection with the skin. The gel-covered surface electrodes must then be precisely placed to ensure electrical activity within the targeted muscle will be received by the electrodes.

EMG techniques have many drawbacks. EMG requires a complex, time-consuming setup procedure, and often requires a specially trained EMG technician in addition to the surgeon performing the surgery. Not only does this add to the time spent in the operating room, it can significantly increase the cost of surgical procedures. Further, surgeons are often resistant to procedures requiring the services of others. In addition to the complex setup, EMG can be an uncomfortable procedure for the patient. Needle electrodes must be driven through the skin and directly into muscle tissue. The needles may increase the risk of infection, and may lengthen the required healing time after the surgical procedure. Moreover, the needles pose an increased risk for medical professionals, due to the potential for accidental needle sticks. Debridement and skin preparation may be an irritant for patients when surface electrodes are used.

Once the electrodes are in place, it is not uncommon for them to come loose and require reattachment. Needle electrodes may be bumped during a surgery, causing them to be displaced from the target region. Surface electrodes, covered with gel, do not adhere strongly to a patient's skin and thus are prone to falling off. When electrodes lose electrical contact with a target muscle, it may not be apparent to the surgeon or EMG technician. Reattaching electrodes, and interpreting issues associated with electrodes, may further lengthen the time required for a surgical procedure, and may lead to additional frustration. Further, reattachment of electrodes during a surgical procedure may risk contamination of the sterile field. Even when EMG electrodes are properly positioned, electrical signals may be difficult to detect, and difficult to interpret. The EMG electrodes are particularly prone to interference. Accordingly, any electrical device within an operating room may affect electrode outputs. This may require a significant amount of work and interpretation to isolate the portion of readings attributable to EMG. When signals are finally received from electrodes, they are often confusing and difficult to interpret. Resulting signals are often very intricate, including various shapes, sizes, frequencies, etc. Accordingly, interpretation of EMG signals may require significant additional training for a surgeon, or may require the services of a specially trained EMG technician, to obtain meaningful information.

In addition to the foregoing, EMG systems may continually provide stimulation to a target nerve to continually monitor electrical activity. Accordingly, when using EMG systems, the muscles innervated by the targeted nerves may continually fire. This may make it difficult to properly restrain a patient, and make surgery more dangerous. It may also prompt electrodes to come loose.

Further, EMG systems which are turned on intermittently during a surgical procedure generally require a delay while a signal is detected and interpreted. This delay prolongs surgical times, and may create a period of risk and uncertainty.

These and other limitations have led to frustration and a lack of confidence in EMG techniques.

### BRIEF SUMMARY

A device, method and system for nerve monitoring are disclosed. The device includes a mechanical sensor such as, but not limited to, an accelerometer, configured to detect a physical response of a muscle or group of muscles in the event that a nerve innervating the muscle or group of muscles responds to a stimulus. The device may also include an indicator which may provide feedback to a user based on at least a portion of an output of the mechanical sensor. The device may be used, for instance, during a surgical procedure to detect proximity to a nerve. In accordance with one exemplary approach, the mechanical sensor includes at least one accelerometer. The accelerometer may be configured to detect muscle motion and/or acceleration.

In accordance with one exemplary approach, a method includes receiving an input from at least one mechanical sensor configured to monitor at least one muscle for a response to a stimulus, and providing a signal representing at least a portion of the input received from the at least one mechanical sensor to a user.

In accordance with one exemplary approach, a system includes a stimulator configured to be positioned within a treatment area. The treatment area may be positioned within a body and may include, or be located near, at least one nerve. The system may also include a mechanical sensor such as, but not limited to, an accelerometer configured to be placed proximate at least one muscle innervated by the at least one nerve. The mechanical sensor may be further configured to monitor the at least one muscle for a response to a stimulus. The system may further include a receiver configured to receive an output from the mechanical sensor, to filter the received output from the mechanical sensor to pass only information indicative of a response to the received stimulus, and to provide an indicator to a user in at least near real time, the indicator indicating whether the at least one muscle is responding to the stimulus.

### BRIEF DESCRIPTION OF THE DRAWINGS

FIG. 1 illustrates an exemplary nerve monitoring device;

FIG. 2 illustrates a connection of exemplary mechanical sensors to a patient;

FIG. 3 illustrates an exemplary nerve monitoring system;

FIG. 4 illustrates a treatment area according to an approach; and

FIG. 5 is an exemplary graph of an output from a mechanical sensor.

### DETAILED DESCRIPTION

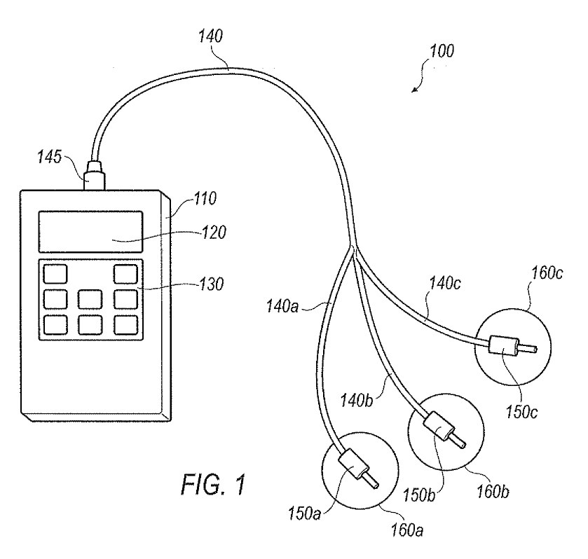

FIG. 1 illustrates an exemplary nerve monitoring device 100, including at least one mechanical sensor 160 in communication with a receiver 110 over a connector such as cable 140. In the illustrated approach, receiver 110 includes a display screen 120, and a user interface including a plurality of buttons 130. In one exemplary approach, a receiver, such as receiver 110, may include a touch screen which may provide information to a user and may also act as a user interface capable of receiving input from a user. Receiver 110 may also include one or more icons, light emitting diodes (LEDs), audible indicators, or other devices configured to provide information to a user. The illustrated cable 140 includes a primary connector 150 connected to receiver 110, and a plurality of secondary connectors 155 removably connected to a plurality of mechanical sensors 160.

The receiver 110 may be a stand alone receiver, as illustrated. It is to be understood, however, that this is by way of example, and not of limitation. A receiver 110 may be included as part of another device, including but not limited to a computer, a personal digital assistant (PDA), or other device. Receiver 110 may be embodied as hardware, as software, or as a combination of hardware and software. Receiver 110 may be configured to receive outputs from the mechanical sensor 160 and to selectively provide an indicator to a user based on at least a portion of the received outputs. An indicator may be a visual and/or audible indicator, which may be used, by way of example and not of limitation, to provide a real-time or near real-time indication of the output received from at least one mechanical sensor 160, or to indicate when the output of at least one mechanical sensor 160 exceeds a predetermined value. A visual indicator may be provided, for example, using a screen, such as screen 120 on receiver 110, on a display incorporated into another device into which receiver 110 is integrated, or a separate display with which receiver 110 may communicate. Audible indicators may be provided, for example, by a speaker (not shown), which may be built in to receiver 110 or provided in another method. Receiver 110 may include one or more user input devices such as, but not limited to, buttons 130, thumb wheels, etc. Buttons 130 may allow a user to interact with receiver 110 to, for example, to edit one or more settings within receiver 110.

The mechanical sensor 160 may be configured to be placed proximate a muscle or group of muscles, and to detect a physical action in a muscle or group of muscles. As used herein, a mechanical sensor 160 may be considered proximate a muscle if the mechanical sensor 160 is sufficiently close to the muscle to register a response upon stimulation of the muscle. The physical action may include, for example, muscle motion, acceleration, displacement, vibration, etc. In one exemplary approach, the mechanical sensor 160 may be an accelerometer. The mechanical sensor 160 may be configured to connect directly to the skin of a patient, in an area proximate a muscle or group of muscles. The mechanical sensor 160 may include an adhesive face to allow the mechanical sensor 160 to be quickly and securely adhered to the patient. The mechanical sensor 160 may be configured to be in electrical contact with the muscle or group of muscles, and/or with the skin to which the mechanical sensor 160 is adhered. Alternatively, the mechanical sensor 160 may be electrically isolated from the muscle or group of muscles and/or the skin to which it may be adhered. As used herein, “electrically isolated” includes being generally isolated from the skin of a patient and/or a muscle located beneath the skin. In any event, embodiments indicated as electrically isolated generally do not have sufficient electrical contact with a particular region to provide an EMG signal. In addition, the mechanical sensor 160 may be compatible with a Magnetic Resonance Imaging (MRI) device, thereby allowing a surgeon to employ mechanical sensor 160 in addition to an MRI device during a surgical procedure. The mechanical sensor 160 may include a connector 150 for removably connecting with a cable, such as cable 140. Cable 140 may transmit an output from mechanical sensor 160 to device 110. As used herein, “MRI compatible” includes being constructed of materials that will not significantly affect readings from an MRI device.

The mechanical sensor 160 may be placed proximate a particular muscle or group of muscles to detect whether the muscle exhibits a physical response to a stimulus. Locations for mechanical sensor 160 may be determined based on the particular surgical procedure. A mechanical sensor 160 may be placed quickly, and may be easily repositioned prior to, or during, a surgical procedure. Mechanical sensor 160 does not pierce the skin, and thus may, but need not, be placed within a sterile field. Further, in one exemplary approach, the mechanical sensor 160 does not require a strong electrical connection with the patient. Accordingly, conductive gel need not be placed between the mechanical sensor 160 and the skin. Moreover, the skin need not be thoroughly cleaned, shaved and debrided, as is required with EMG connections. This allows connectors to be attached quickly, and greatly improves reliable adhesion of sensors 160. Furthermore, when a muscle exhibits a physical response to a stimulus, a corresponding response is exhibited not only by the skin directly above the target muscle, but also by the skin in the same general area of the muscle. Thus, whereas EMG electrical sensors must be placed precisely to ensure reliable reading of electrical signals from a target muscle, mechanical sensors 160 need only be placed in the general area of the target muscle. This allows improved reliability, with improved ease of use.

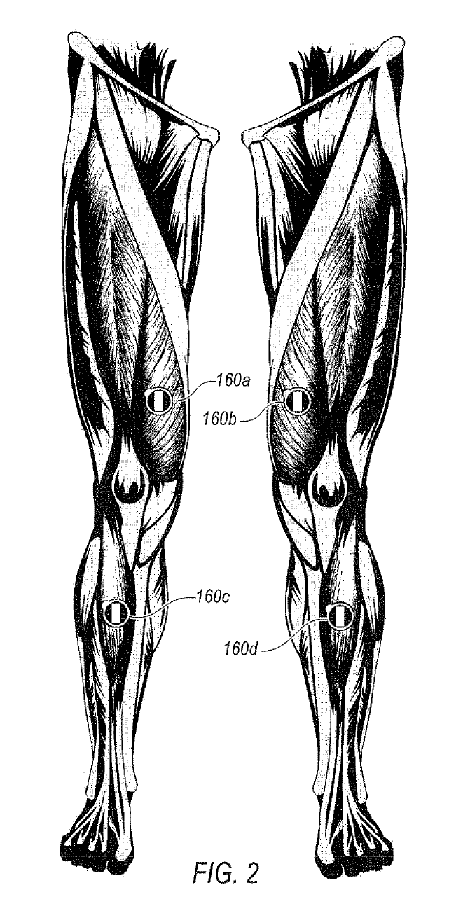

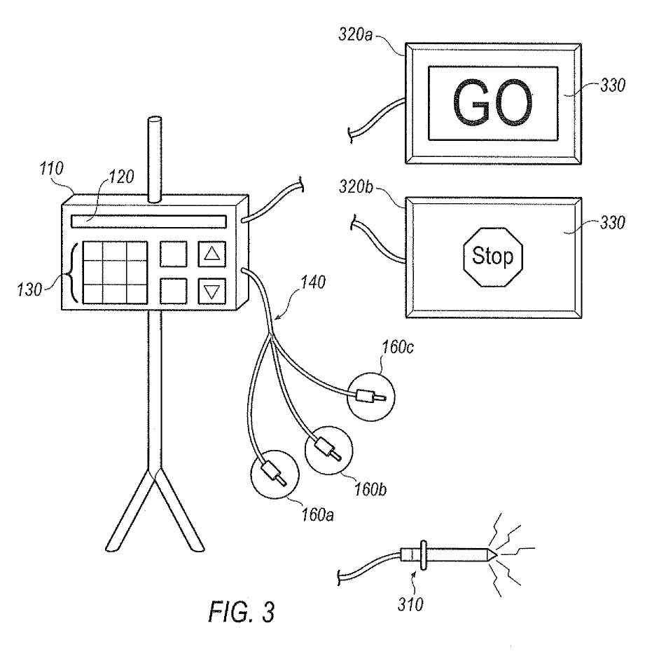

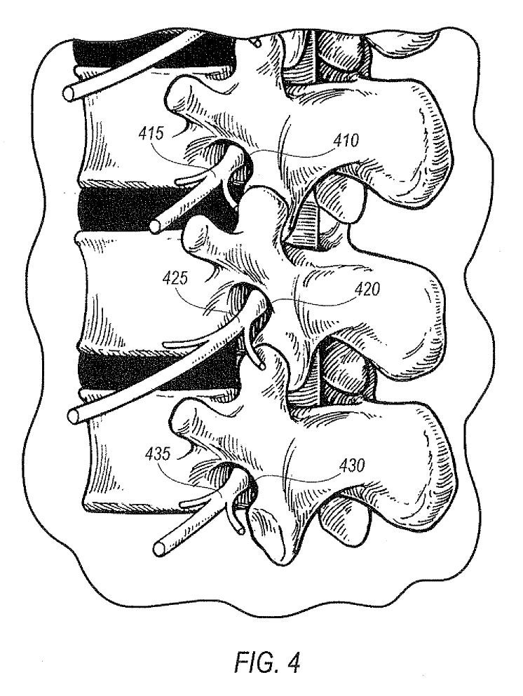

Referring now to FIGS. 2 and 4, FIG. 2 illustrates an exemplary placement of a series of sensors 160 on the legs of a patient. The illustrated sensor placement is meant as an exemplary approach, and is in no way intended to be limiting. FIG. 4 illustrates an exemplary treatment area 400. The illustrated sensor placement may be useful, for example, for monitoring nerves exiting the L2, L3 and L4 foramen (410, 420, 430, respectively,) during a surgical procedure. By way of example, and not of limitation, during a discectomy of the lumbar spine a surgeon may know that the nerves 415, 425 and 435 exiting the L2, L3 and L4 foramen 410, 420, 430 are potentially located in the treatment region 400. The surgeon may then place mechanical sensors 160 on muscles innervated by those nerves 415, 425, 435. For instance, in the illustrated approach, mechanical sensors 160a and 160b are placed on the vastus medialis muscles, which are innervated by the nerves, such as nerves 415 and 425 exiting the L2 and L3 foramen 410, 420, and sensors 160c and 160d are placed on the tibialis anterior muscles, which are innervated by the nerves, such as nerve 435, exiting the L4 foramen 430. During the surgical procedure, the surgeon may provide a stimulus within a treatment region, such as treatment region 400. The treatment region may be specific to a particular surgical procedure. For instance, a treatment region may include the area which a surgeon may generally access during a particular surgery. The treatment region may be within the body of a patient (intracorporeal), outside the body, on the surface of the body, such as on the skin of the patient, or any combination thereof. The stimulus may be, for example, an electrical charge. The stimulus may be provided through the insertion of a stimulator, such as stimulator 310 (FIG. 3, described below). Alternatively, a stimulus may be provided by one or more medical instruments typically used for a surgical procedure, such as an endoscope device, a scalpel, etc. A stimulus may be provided constantly during a surgical procedure, or may be selectively delivered by a surgeon. That is, a surgeon may provide a stimulus intermittently during a surgical procedure to a treatment area.

If a nerve is near the provided stimulus, the stimulus will be received by a nerve. Upon receiving the stimulus, the nerve may induce a physical response in the muscles, such as motion, acceleration, displacement, vibration, etc. This muscle response may be registered by one or more mechanical sensors 160. The response may then trigger an output from one or more mechanical sensors 160 which may be transmitted over cable 140 to device 110.

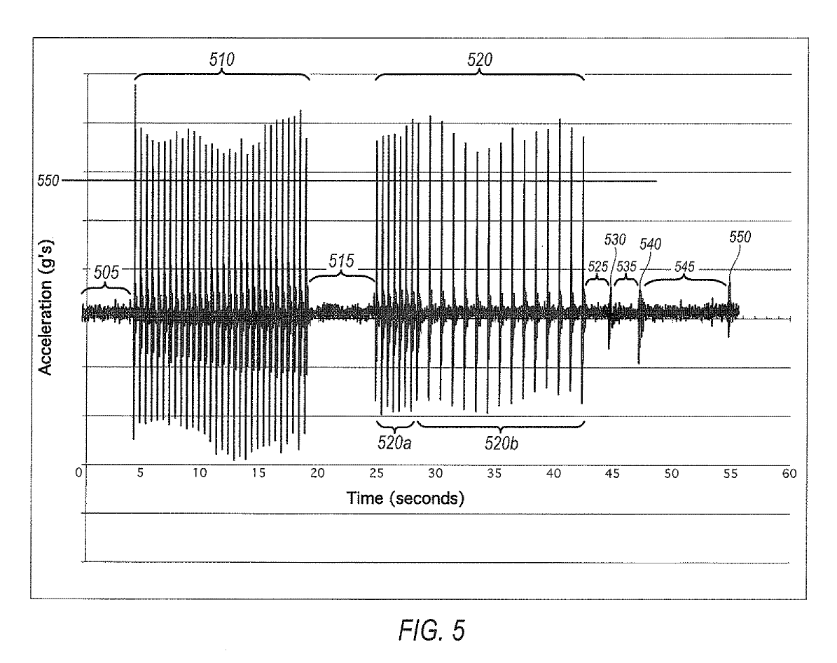

Receiver 110 may provide a response to a user, such as over display screen 120, based on the signal received from the mechanical sensor 160. For example, receiver 110 may provide a graphical representation, such as graph 500 (FIG. 5,) or a numerical representation of the output of the mechanical sensor 160, a “Go/No Go” display, or other visual display. A “Go/No Go” style display may, for example, provide a first indication, such as the word “Go,” a green light, a “thumbs up,” or other indication when the output of the mechanical sensor is within a first range, and may provide a second indication, such as the words “No Go,” a red light, a “thumbs down,” or other indication when the output of the mechanical sensor is, for example, within a second range, or above a threshold value. Additionally, or alternatively, receiver 110 may be configured to provide an audible alert to a user. An alert may be provided, for example, if the output of the mechanical sensor 160 exceeds a certain value. Alternatively, an audible signal may be provided throughout a procedure and may change based on the received output of the mechanical sensor 160. For instance, an alert may sound with increased regularity, at an increased frequency, at a greater volume, etc., as increased activity is detected by the mechanical sensor 160. A user interface, such as buttons 130, may allow a user to interact with the receiver 110 in order to set values, such as threshold values, and/or to format one or more parameters. Parameters may include parameters related to the device, mechanical sensors 160, displays, stimulators, or other elements as may be known.

The receiver 110 may receive an output from the one or more mechanical sensors 160. Receiver 110 may, for instance, compare the received output to a threshold value, to determine whether the output exceeds the threshold value. Additionally, or alternatively, receiver 110 may provide the user with a representation of the output of the one or more mechanical sensors 160. In one embodiment, receiver 110 may provide the user with a graphical representation of the output of the one or more mechanical sensors 160, such as graph 500 (FIG. 5).

FIG. 3 illustrates an exemplary system 300 for nerve monitoring. System 300 includes a plurality of mechanical sensors 160 which may communicate with a receiver 110 over a series of cables 140. System 300 includes a stimulator 310, configured to provide a stimulus within a treatment area. Receiver 110 is in communication with a display 320. Display 320 is configured to communicate information related to the output of at least one mechanical sensor 160 using a screen 330. System 300 illustrates display 320 in a first state 320a, and a second state 320b. Display 320a includes a graphic on screen 330 which may be used to indicate that the output received from mechanical sensors 160 is below a certain threshold value, such as threshold 550 (FIG. 5.) Display 320b includes a graphic on screen 330 which may be used to indicate, for instance, that the output received from at least one mechanical sensor 160 is above a particular threshold value 550 (FIG. 5).

FIG. 5 illustrates a graphical representation of the output of a mechanical sensor 160 during an exemplary approach. In the exemplary approach illustrated in FIG. 5, mechanical sensor 160 is an accelerometer. Graph 500 represents the output of a first accelerometer 160 over a period of time, encompassing a number of distinct regions. In the first region, 505, the mechanical sensor 160 has been placed on the patient though at this time, the nerve or nerves innervating the target muscle or muscle group has not been stimulated. This may indicate that it is safe to proceed or continue with a procedure. In the second region, 510, a stimulus has been provided near the target nerve. The stimulus provided during this second region 510 may be sufficiently large and/or sufficiently near a nerve that the stimulus elicits a response in the nerve. The exemplary stimulus provided during the second region 510 is an electrical signal having a frequency of approximately 2 Hz, although it is to be understood that other stimuli may be used. A corresponding response is registered by the mechanical sensor 160, the output of which is displayed on graph 500. The illustrated response has a frequency similar to the frequency of the provided stimulus. The magnitude of the illustrated response of the second region 510 is greater than the threshold value 550. In the illustrative example, a particular threshold value 550 was selected, though it is to be understood that other magnitudes may be selected, as desired. In the third region, 515, the stimulus is no longer being registered by the nerve and accordingly, the output from the accelerometer 160 returns to near zero. This may signify to a surgeon that it is again safe to continue with a medical procedure. In the fourth region, 520, the stimulus is again received by a nerve, causing a corresponding physical response near the target region. The stimulus provided during the fourth region is altered from a 2 Hz electrical signal (520a) to a 1 Hz electrical signal (520b), causing a corresponding change in the reaction of the innervated muscle and a mechanical sensor 160 connected thereto. While the response registered by mechanical sensor 160 in region 520a has a frequency greater than the response registered in region 520b, the magnitude of the registered response still exceeds the threshold value 550. The stimulus is again removed in the fifth, seventh and ninth regions, 525, 535 and 545. The sixth and eighth regions, 530 and 540, illustrate an exemplary response registered by the mechanical sensor 160 to a stimulus other than the excitation of a nerve. For example, in the exemplary regions 530 and 540, the operating table may have been bumped by the surgeon. As illustrated in graph 500, the response registered by a mechanical sensor 160 to a stimulus provided to a nerve may be significantly greater than the response registered from another stimulus. Accordingly, a “Go/No Go” style display may display “No Go” when a response registered by a mechanical sensor 160 is above threshold 550, such as during regions 510 and 520, and may display “Go” when a response is not registered from a mechanical sensor 160, or when a response registered by a mechanical sensor 160 is below threshold 550, such as during regions 505, 515, 525, and thereafter.

While graph 500 illustrates the output of a single mechanical sensor 160, it is to be understood that this is by way of example and not of limitation, and a graph may include representations of the output of multiple mechanical sensors 160. Moreover, a display such as display 120, may illustrate the output of one or more mechanical sensors 160, one or more “Go/No Go” signals related to one or more mechanical sensors 160, or other information.

A stimulator may be a stand-alone device, or may alternatively be incorporated into a medical instrument, such as a pedicle probe, needle, guide wire, dilator, retractor, independent multiprobe, elevator, etc. The stimulator may provide a stimulus, for example, along a distal point of the stimulator. The stimulus may include an electrical signal which may energize, for example, the area around a distal tip of the stimulator. Alternatively, the stimulus may be a physical stimulus, which may provided by a stimulator physically contacting a nerve. According to one exemplary approach, a stimulator may provide a constant stimulus throughout a surgical procedure. In such an approach, a response may be registered by a mechanical sensor 160 when the stimulus is provided proximate a nerve innervating a muscle located proximate the mechanical sensor 160. Alternatively, a stimulator may provide a stimulus intermittently, such as at a regular interval, which may be predetermined, or may be provided selectively, such as upon request by a surgeon. The surgeon may monitor the output of the mechanical sensor 160 and may thereby determine whether the stimulator is located proximate a nerve. A stimulus may be considered proximate a nerve if the stimulus is near enough the nerve to elicit a response in the nerve.

In one exemplary approach, a surgeon may identify a first treatment region in which to begin a surgical procedure. Throughout the surgical procedure the surgeon may stimulate the area in which the surgeon is working, while monitoring the output of at least one mechanical sensor 160. If at any point there is a response registered by a mechanical sensor 160, the surgeon may temporarily pause the procedure. The surgeon may determine, based on the registered response, whether it is safe to continue the procedure in the present location. The surgeon may determine whether it is safe by, for instance, viewing the magnitude of the registered response, based on whether the response is a “Go” or a “No Go” response, etc. If the surgeon determines that it is not safe to continue in the present location, the surgeon may determine another location at which to continue the procedure. For instance, the surgeon may approach an area from a different angle, using a different treatment method, or otherwise alter the surgery. The surgeon may determine the safety of a subsequent method or approach by stimulating the proposed area, and monitoring a mechanical sensor 160. Additionally or alternatively, a surgeon may stimulate one or more areas within, or near, a proposed treatment region in an effort to identify or locate nerves prior to, or during, a surgical procedure.

Although exemplary embodiments of the mechanical sensor 160 have generally included an accelerometer, it is to be understood that this is by way of example and not of limitation. A mechanical sensor may include other types of mechanical sensors or motion sensors, as desired. Additionally, a mechanical sensor 160 may include more than one sensor, which may, but need not, be the same type of sensor.

The preceding description has been presented only to illustrate and describe exemplary embodiments of the methods and systems of the present invention. It is not intended to be exhaustive or to limit the invention to any precise form disclosed. It will be understood by those skilled in the art that various changes may be made and equivalents may be substituted for elements thereof without departing from the scope of the invention. In addition, many modifications may be made to adapt a particular situation or material to the teachings of the invention without departing from the essential scope. Therefore, it is intended that the invention not be limited to the particular embodiment disclosed as the best mode contemplated for carrying out this invention, but that the invention will include all embodiments falling within the scope of the claims. The invention may be practiced otherwise than is specifically explained and illustrated without departing from its spirit or scope. The scope of the invention is limited solely by the following claims.

What is claimed is:

1. A device, comprising:
a mechanical sensor configured to monitor at least one muscle for a response to a stimulus; and
an indicator configured to provide feedback to a user based on at least a portion of an output of the mechanical sensor.

2. The device of claim 1, wherein the stimulus is provided within a treatment area, the treatment area including at least one nerve, the nerve innervating the at least one muscle.

3. The device of claim 1, wherein the mechanical sensor includes at least one accelerometer configured to detect muscle motion in response to a stimulus.

4. The device of claim 1, wherein the mechanical sensor is configured to be placed proximate the at least one muscle prior to a medical procedure.

5. The device of claim 4, wherein the mechanical sensor includes an adhesive face.

6. The device of claim 4, wherein the mechanical sensor is electrically isolated from the muscle.

7. The device of claim 1, further comprising a stimulator configured to provide a stimulus within a treatment area.

8. The device of claim 7, wherein the stimulator is configured to provide an electrical stimulus.

9. The device of claim 8, wherein the stimulator is incorporated into a medical device.

10. The device of claim 1, wherein the at least one mechanical sensor is MRI-compatible.

11. The device of claim 1, wherein the indicator indicates at least whether a portion of the output of the mechanical sensor is within a predetermined range of values.

12. The device of claim 11, wherein the feedback is a binary output indicating whether the output of the mechanical sensor is, or is not, above a predetermined value.

13. The device of claim 1, further comprising a filter configured to block a portion of the mechanical sensor output that is indicative of movement by something other than the muscle.

14. A method, comprising:
receiving an input from at least one mechanical sensor;
the at least one mechanical sensor configured to monitor at least one muscle for a response to a stimulus; and
providing a signal to a user, the signal indicative of at least a portion of the input received from the at least one mechanical sensor.

15. The method of claim 14, wherein the stimulus is an electrical signal provided to a nerve, the nerve innervating the at least one muscle.

16. The method of claim 14, wherein the stimulus is a mechanical signal provided to a nerve, the nerve innervating the at least one muscle.

17. The method of claim 14, wherein providing a signal to a user includes providing a signal indicating whether a medical device is proximate a nerve.

18. The method of claim 14, wherein the mechanical sensor includes at least one accelerometer configured to detect muscle motion in response to the stimulus.

19. The method of claim 14, further comprising positioning a mechanical sensor proximate at least a first muscle.

20. The method of claim 14, further comprising inserting at least a portion of a stimulator within a treatment area, the stimulator configured to selectively provide a stimulus to at least one nerve when the stimulator is positioned proximate the at least one nerve.

21. The method of claim 14, further comprising filtering the received input.

22. The method of claim 21, wherein filtering the received input includes passing only a portion of the received input indicating a response to the received stimulus.

23. The method of claim 14, wherein the signal includes a signal indicating when a stimulator is proximate the at least one nerve.

24. The method of claim 14, wherein the signal to a user includes a signal indicating whether it is safe to continue with a medical procedure.

25. A system comprising:
a stimulator configured to be positioned within a treatment area, the treatment area positioned within a body and proximate at least one nerve;
a mechanical sensor configured to be placed proximate at least one muscle, the at least one muscle innervated by the at least one nerve;
the mechanical sensor further configured to monitor the at least one muscle for a response to a stimulus; and
a receiver configured to receive an output from the mechanical sensor, to filter the received output from the mechanical sensor to pass only information indicative of a response to the received stimulus; and to provide an indicator to a user in at least near real time, the indicator indicating whether the at least one muscle is responding to the stimulus.
